(19) United States
(12) Patent Application Publication
(10) Pub. No.: US 2013/0042875 A1
(43) Pub. Date: Feb. 21, 2013

(54) TONGUE RETAINING DEVICE

(71) Applicant: Craig Schwimmer, Dallas, TX (US)

(72) Inventor: Craig Schwimmer, Dallas, TX (US)

(73) Assignee: SNORING CENTER USA, LLC, Dallas, TX (US)

(21) Appl. No.: 13/210,574

(22) Filed: Aug. 16, 2011

(51) Int. Cl. A61F 5/56; A61C 5/14

(52) U.S. Cl. 128/848; 128/860

(57) ABSTRACT

A tongue retaining device and a method of using the same are provided. The device includes an implant member configured to be anchored in a tongue, a dental plate configured to be worn upon mandibular teeth, and a coupling member couplable between the implant member and the dental plate to promote direct anterior displacement of the tongue. The tongue retaining device may be offered as part of a kit with a tongue-piercing device.

### BACKGROUND

Sleep disordered breathing, including snoring and Obstructive Sleep Apnea (OSA), adversely affects millions of people. In many cases, sleep disordered breathing is caused when soft tissue in an upper airway vibrates during respiration while a person is asleep. While relatively minor vibration of this soft tissue results in simple snoring, severe vibration may cause the airway to be compromised or repeatedly collapse, which results in OSA.

### BRIEF DESCRIPTION OF THE DRAWINGS

The accompanying drawings incorporated in and forming a part of the specification illustrate several aspects of the present disclosure and, together with the description, serve to explain the principles of the present disclosure. In the drawings:

FIG. 1 is a perspective view of the tongue retaining device prior to assembly;

FIG. 2 is a perspective view of the tongue retaining device of FIG. 1 packaged with a tongue-piercing device and offered as a tongue retaining device kit;

FIG. 3 is a method of promoting direct anterior displacement of a tongue as a treatment for sleep disordered breathing with the tongue retaining device of FIG. 1;

FIG. 4 is a perspective view of an implant member from the tongue retaining device of FIG. 1 being anchored in a tongue with the tongue-piercing device of FIG. 2;

FIG. 5 is a simplified cross section view of the dental plate from the tongue retaining device of FIG. 1 being molded on mandibular teeth;

FIG. 6 is a simplified side elevation view of the coupling member of the tongue retaining device of FIG. 1 coupling the implant member and dental plate; and

FIG. 7 is a simplified top view of the coupling member of the tongue retaining device of FIG. 1 coupling the implant member and dental plate.

### DETAILED DESCRIPTION OF THE DRAWINGS

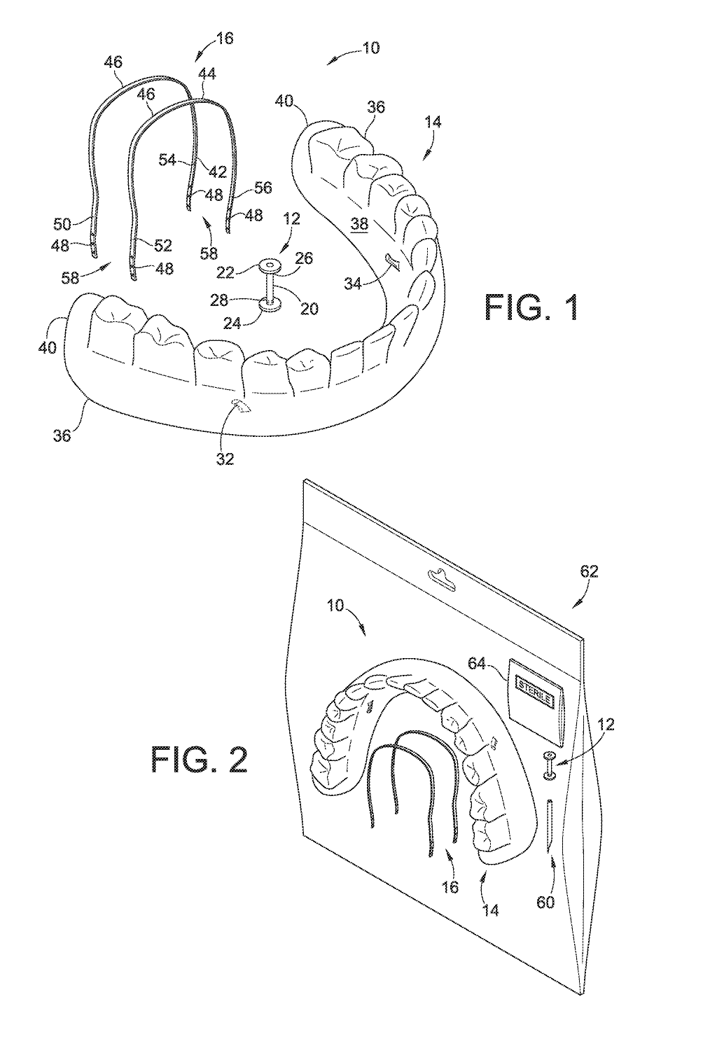

Referring to FIG. 1, a tongue retaining device 10 is illustrated. As will be more fully explained below, the tongue retaining device 10 promotes and achieves direct anterior displacement of the tongue as a treatment for sleep disordered breathing (e.g., snoring and Obstructive Sleep Apnea).

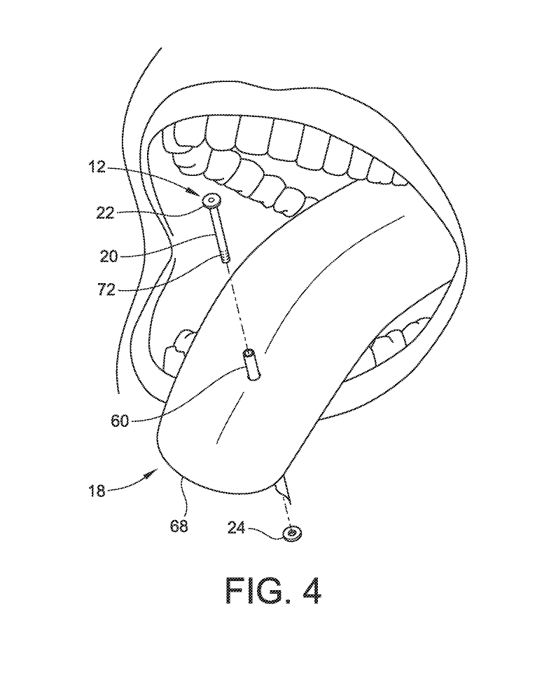

As depicted in FIG. 1, the tongue retaining device 10 generally comprises an implant member 12, a dental plate 14, and a coupling member 16. The implant member 12 is configured to be anchored in a human tongue 18 (see FIG. 4). In some embodiments, the implant member 12 is a grommet, stud, barbell, rivet, or other similar device. The implant member 12 generally includes a post 20 interposed between an upper lip 22 and a lower lip 24. As shown, the upper lip 22 is generally integrally formed with, or secured to, an upper portion 26 of the post 20. The lower the lower lip 24 is generally releasably secured to a lower portion 28 of the post 20. In one embodiment, the lower lip 24 is threadably engaged with the lower portion 28 of the post 20. As such, the lower lip 24 and lower portion 28 of the post 20 can be selectively coupled or uncoupled, depending on the direction of rotation of one relative to the other.

As shown in FIG. 1, the post 20 is formed from a relatively thin, straight, hollow tube. However, in some embodiments the post 20 may be curved, undulating, or otherwise non-linear. In addition, the post 20 may be partially or entirely solid. The post 20 is generally provided with a length somewhat greater than a thickness of the tongue 18.

Despite the upper and lower lips 22, 24 being depicted as generally flat discs in FIG. 1, the upper and lower lips can have other configurations or be formed in other suitable shapes. For example, in some embodiments the upper and lower lips 22, 24 may resemble a heart, a star, or some other two-dimensional shape when viewed from above or below. In addition, the upper and lower lips 22, 24 may also resemble a sphere, a hemisphere, or have some other three-dimensional shape. In some embodiments, the upper and lower lips 22, 24 may be free of an aperture or be partially or entirely solid.

The implant member 12 may be formed from a variety, or combination of, different materials. For example, the implant member 12 may be made from stainless steel, titanium, acrylic, and plastic. In some embodiments, the implant member 12 is colored to resemble a color of the tongue 18 (i.e., flesh colored). In some embodiments, the implant member 12 is all or partially formed from a transparent material such that the natural color of the tongue 18 may be perceived through the implant member 12.

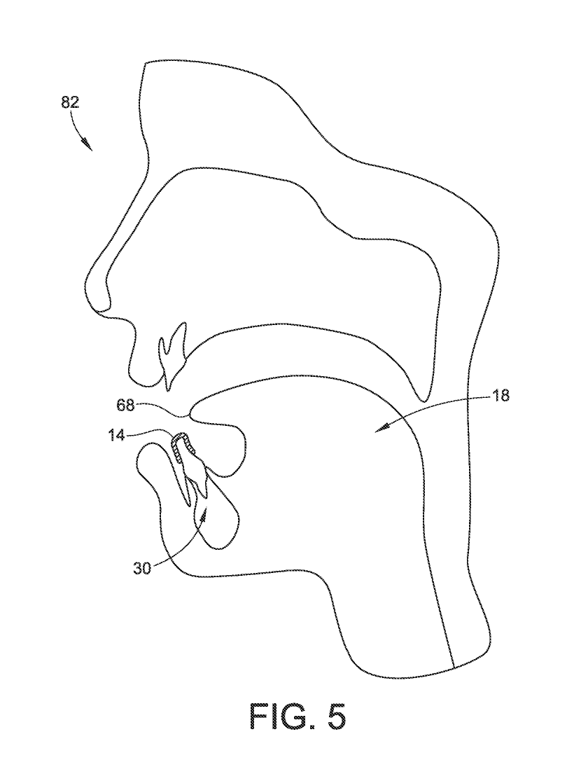

The dental plate 14 is configured to be comfortably fitted over and worn upon the mandibular teeth 30 (see FIG. 5) of a person. Still referring to FIG. 1, the dental plate 14 generally supports a first hook 32 and a second hook 34 on opposing sides 36 of the dental plate 14. As shown, the pair of hooks 32, 34 are laterally-spaced apart from each other, disposed on an inner face 38 of the dental plate 14, and situated forward of a posterior 40 of the dental plate.

While a pair of hooks 32, 34 is depicted in FIG. 1, one or more hooks may be provided on the dental plate 14. In addition, while the hooks 32, 34 are illustrated as projecting from the inner face 38 of the dental plate 14 forward of the posterior 40, the hooks may be otherwise arranged. In some embodiments, the dental plate 14 may be formed from a soft, moldable plastic.

As will be more fully explained below, the coupling member 16 is generally couplable between the implant member 12 and the dental plate 14 to promote direct anterior displacement of the tongue. In some embodiments, the coupling member 16 may be a lone elastic band or several bands (e.g., more than two) working together. In addition, in some embodiments the coupling member 16 may be a resilient or elastic member formed from bands, cords, and the like. In some embodiments, the coupling member 16 may be formed from a pair cooperating magnets with one of the magnets supported by the implant member 12 and the other magnet supported by the dental plate 14. In some embodiments, the coupling member 16 may be other tension-providing or coupling devices or mechanisms (e.g., thin metal wire, etc.).

In some embodiments, and as illustrated in FIG. 1, the coupling member 16 includes an upper band 42 and a lower band 44. To couple the coupling member 16 to the implant member 12, the upper band 42 engages or encircles the upper portion 26 of the post 20 of the implant member 12. Similarly, the lower band 44 engages or encircles a lower portion 28 of the post 20. In some embodiments, a central portion 46 of the upper and lower bands 42, 44 is biased against the post 20 of the implant member 12 when the tongue retaining device 10 is assembled and in use.

To releasably secure the coupling member 16 to the dental plate 14, the first hook 32 is inserted through an aperture 48 on a first end 50 of the upper band 42 and an aperture 48 on a first end 52 of the lower band 44 and the second hook 34 is inserted through an aperture 48 on a second end 54 of the upper band 42 and an aperture 48 on the second end 56 of the lower band 44.

While FIG. 1 depicts the coupling member 16 having apertures 48 and the dental plate 14 having hooks 32, 34, other structures or methods of attachment may be used to couple the coupling member 16 to the dental plate 14. For example, in some embodiments the dental plate 14 may include one or more slots of decreasing width, each slot receiving the distal end of an elastic band. As tension is added to the elastic band, the distal end of the elastic band is drawn farther into the slot. Due to the decreasing width of the slot, the slot compresses or squeezes the distal end of the elastic band to provide a secure, but releasable, connection. In some embodiments, other fasteners, fastening devices, or fastening methods may be adapted and used to couple the coupling member 16 to the dental plate 14.

Still referring to FIG. 1, the upper band 42 and the lower band 44 each include an adjustment feature 58 to selectively change an amount of tension supplied by the upper and lower bands 42, 44. As shown in FIG. 1, the adjustment feature 58 comprises a set or plurality of the apertures 48 spaced-apart from one another along a length of the upper and lower bands 42, 44. The upper and lower bands 42, 44 can be stretched to place a selected one of the apertures 48 upon the hooks 32, 34 disposed on the dental plate 14.

The upper band 42 and the lower band 44 may be trimmed with a cutting device (e.g., a scissors) to reduce a length thereof. For example, if one of the hooks 32, 34 is placed through a center aperture on the coupling member 16, the cutting device may be used to trim off the end of the coupling member 16 containing the aperture 48 closest to the distal end of the upper and lower bands 42, 44.

As illustrated in FIG. 2, the tongue retaining device 10 may be packaged, offered for sale, and sold with a tongue-piercing device 60 as a tongue retaining device kit 62. In some embodiments, the tongue-piercing device 60 included with the kit 62 may be a hollow needle, a cannula needle, or another needle suitable for piercings. In some embodiments, the tongue-piercing device 60 may be a hollow 14 gauge needle. In some embodiments, the kit 62 may include a sterilizer 64, such as a sterile alcohol swab or wipe for sterilizing a surface or area prior to piercing, local anesthetic, or other supplies.

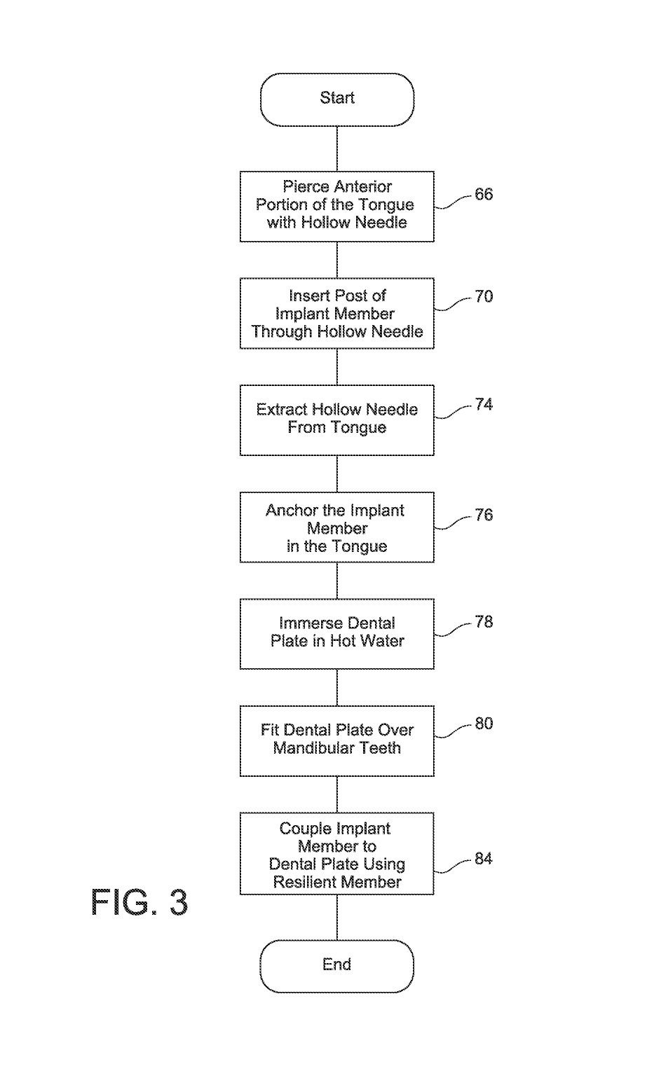

Referring now to FIGS. 3-7, a method of promoting direct anterior displacement of a tongue as a treatment for sleep disordered breathing is disclosed. At block 66 of FIG. 3 and as illustrated in FIG. 4, an anterior portion 68 of the tongue 18 is pierced using the tongue piercing device 60, which is illustrated as a 14 gauge hollow needle. In some embodiments, the tongue 18 is pierced in the midline in the mobile or anterior segment distal to the lingual frenulum.

At block 70, the post 20 from the upper portion of the implant member 12 is inserted through the hollow needle until a distal end 72 of the post 20 is exposed beneath the tongue 18. At block 74, the hollow needle is extracted from the tongue 18. As illustrated in FIG. 4, the hollow needle is drawn downwardly through the musculature of the tongue 18 in such a manner as to leave the post 20 of the implant member 12 embedded in the tongue 18 after extraction of the needle. In some embodiments, a solid needle is used to pierce the tongue and is extracted from the tongue 18 before the implant device 12 is inserted.

Now that the post 20 of the implant member 12 is exposed beneath the tongue 18 and the needle has been removed, at block 76 the lower lip 24 of the implant member 12 is secured to the distal end 72 of the post 20 to anchor the implant member 12 in the tongue 18. In some embodiments, the lower lip 24 is secured to the distal end of the post 20 using mating threads on the post 20 and the lower lip 24 as shown in FIG. 3.

Next, if the dental plate has not previously been prepared, at block 78 the dental plate 14 of FIG. 1 is immersed in hot water to make the dental plate pliable or moldable. At block 80, the dental plate 14 is removed from the hot water and, while still sufficiently warm, fitted over and onto mandibular teeth 30 of a patient 82 as illustrated in FIG. 5.

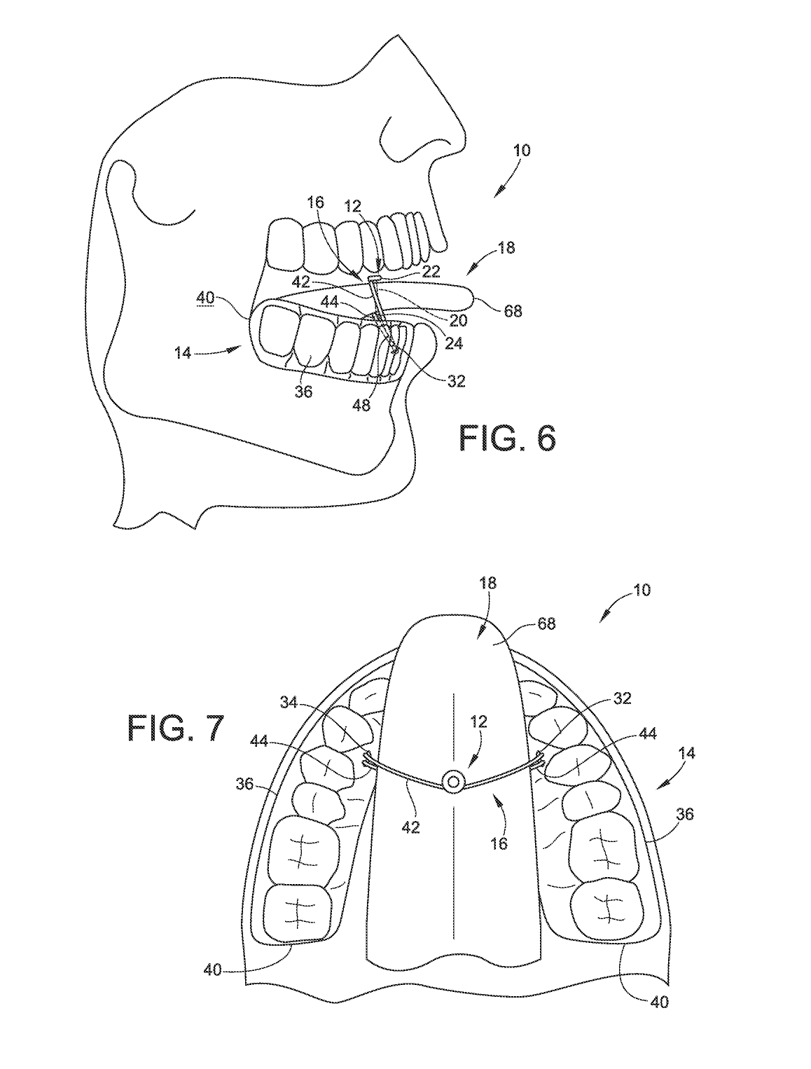

Thereafter, at block 84 the implant member 12 is coupled to the dental plate 14 worn on mandibular teeth 30 using the coupling member 16. In some embodiments, the implant member 12 and the dental plate 14 are coupled together by placing an upper band 42 of the coupling member 16 around the upper portion 26 of the post 20 and the lower band 44 around the lower portion 28 of the post 20 as illustrated in FIGS. 6-7. Thereafter, the first end 50 of the upper band 42 and the first end 52 of the lower band 44 are releasably secured to the first hook 32 on the dental plate 14 as shown in FIG. 7. Likewise, the second end 54 of the upper band 42 and the second end 56 of the lower band 44 are releasably secured to the second hook 34 on the dental plate 14 to couple the implant member to the dental plate 14.

In the configuration described above, the upper band 42 extends from one side 36 of the dental plate 14, around the upper portion 26 of the post 20 proximate the top surface of the tongue 18, and to the other side 36 of the dental plate 14. In addition, the tower band 44 extends from one side 36 of the dental plate 14, around the lower portion 28 of the post 20 proximate the bottom surface of the tongue 18, and to the other side 36 of the dental plate 14.

From the foregoing, those skilled in the art will recognize and appreciate that the tongue retaining device 10 promotes and achieves direct anterior displacement of the tongue as a treatment for sleep disordered breathing.

What is claimed is:

1. A tongue retaining device, comprising:
an implant member configured to be anchored in a tongue;
a dental plate configured to be worn upon mandibular teeth; and
a coupling member couplable between the implant member and the dental plate to promote direct anterior displacement of the tongue.

2. The tongue retaining device of claim 1, wherein the implant member comprises a post interposed between an upper lip and a lower lip, the lower lip threadably securable to the post.

3. The tongue retaining device of claim 1, wherein the implant member is colored to resemble a color of the tongue.

4. The tongue retaining device of claim 1, wherein the implant member is formed from a transparent material.

5. The tongue retaining device of claim 1, wherein the implant member comprises a post interposed between an upper lip and a lower lip and the coupling member comprises an upper band and a lower band, the upper band configured to engage an upper portion of the post and the lower band configured to engage a lower portion of the post.

6. The tongue retaining device of claim 5, wherein the upper band and the lower band each include an adjustment feature to selectively change an amount of tension supplied thereby.

7. The tongue retaining device of claim 6, wherein the adjustment feature is a series of spaced-apart apertures, each of the apertures configured to selectively receive a hook disposed on the dental plate.

8. The tongue retaining device of claim 5, wherein at least one of the upper band and the lower band is configured to be trimmed with a cutting device to reduce a length thereof.

9. The tongue retaining device of claim 1, wherein the coupling member comprises a set of resilient members, at least one of the set of resilient members having an elasticity different than another of the set of resilient members.

10. A tongue retaining device kit, comprising:
an implant member configured to be anchored in a tongue;
a dental plate configured to be worn upon mandibular teeth;
a coupling member couplable between the implant member and the dental plate to promote direct anterior displacement of the tongue; and
a device configured to pierce the tongue.

11. The tongue retaining device kit of claim 10, wherein the implant member comprises a post interposed between an upper lip and a lower lip, the lower lip threadably securable to the post.

12. The tongue retaining device kit of claim 10, wherein the coupling member comprises an upper band and a lower band, the upper band configured to engage an upper portion of a post and the lower band configured to engage a lower portion of the post.

13. The tongue retaining device kit of claim 11, wherein the dental plate includes a first hook laterally spaced-apart from a second hook, and wherein a first end of the upper band and a first end of the lower band are configured to be releasably secured to the first hook and a second end of the upper band and a second end of the lower band are configured to be releasably secured to the second hook.

14. The tongue retaining device kit of claim 10, wherein the device configured to pierce the tongue comprises a hollow needle.

15. A method of promoting direct anterior displacement of a tongue as a treatment for sleep disordered breathing, the method comprising:
piercing an anterior portion of the tongue using a needle;
inserting a post from an upper portion of an implant member through the tongue until a distal end of the post is exposed;
extracting the needle from the tongue;
securing a lower lip to the distal end of the post to anchor the implant member in the tongue;
coupling the implant member to a dental plate worn on mandibular teeth using a coupling member to promote direct anterior displacement of the tongue.

16. The method of claim 15, wherein the lower lip is secured to the distal end of the post by threadably securing the lower lip to the distal end of the post.

17. The method of claim 15, wherein the method further comprises molding the dental plate around the mandibular teeth.

18. The method of claim 17, wherein the method further comprises immersing the dental plate in hot water prior to molding the dental plate.

19. The method of claim 15, wherein the method further comprises placing an upper band of the coupling member around an upper portion of the post and a lower band of the coupling member around a lower portion of the post to couple the implant member to the dental plate.

20. The method of claim 19, wherein the method further comprises releasably securing a first end of the upper band and a first end of the lower band to a first hook on the dental plate and a second end of the upper band and a second end of the lower band to a second hook on the dental plate to couple the implant member to the dental plate.
